(19) United States
(12) Patent Application Publication
(10) Pub. No.: US 2005/0069500 A1
(43) Pub. Date: Mar. 31, 2005

(54) Masticatory-releasable dentifrice article

(76) Inventor: Todd A. Bland, Omaha, NE (US)

Correspondence Address: Todd A. BLAND, 13708 Frederick Avenue, Omaha, NE, 68138, US

(21) Appl. No.: 10/670,853

(22) Filed: Sep. 26, 2003

(51) Int. Cl. A61K 7/16

(52) U.S. Cl. 424/49

(57) ABSTRACT

Readily retrievably selectable from commercially available dentifrices, a selected dentifrice is solid-state dosage charged within a relatively-thin readily-masticatable and hydroscopic/salivascopic envelope material. The envelope material is preferably of conventional normally-solid thermoplastic gelatins, readily commercially available in various forms such as, inter alia, chargeably-receiveable capsules, in laminatable thin-sheets, etc. Plural such dentifrice-enveloped articles can be assembled into a storage vessel and thence periodically retrieved individually at subsequent teethbrushing sessions.

### BACKGROUND OF THE INVENTION

Well recognized in the prior art is the conventional tooth-brushing technique which entails the user's periodic discharging of a judiciously appropriate dosage from a receptacle supply (e.g. from a squeexable-tube, a sprinkleable-can, etc.) of amorphous dentifice onto a manually wieldable toothbrush. However, such conventional toothbrushing technique is inadequate for many toothbrushers (e.g. some children, some manually deficient adults, etc.) in so far as such mentioned persons lack the maturity and/or the ability to manually discharge an appropriate dosage of amorphous dentifrice from its receptacle supply onto a selectable toothbrush. As a result, many such mentioned indiscriminately handicapped persons are likely to charge an inappropriate dentifrice dosage (either insufficient, and/or exceedingly wasteful) for a periodic toothbrushing session.

### GENERAL OBJECTIVE OF THE PRESENT INVENTION

In view of the prior art toothbrushing techniques deficiency alluded to hereabove, and markedly applied to (but not exclusively for) children toothbrushers, it is the General Objective of the Present Invention to enable such children, and others similarly situated, to judiciously appropriately discharge from receptacles of amorphous dentifrice onto the user's toothbrush at each periodic toothbrushing session. And among various other ancillary objectives, to allure such children (and others)onto their toothbrushing duties. Among, but not limited to, said (and others) are physically handicapped persons, and persons lacking in-door plumbing facilities such as campers, soldiers in the field, and the like.

### GENERAL STATEMENT OF THE INVENTION

With the aforementioned General Objective of the Present Invention in view, and together with other ancillary and related objectives which will become more apparent as the ensuing Detailed Drawing Description proceeds, the Masticatory-Releasable Dentifrice Article of the present invention generally comprises: a solid-state dentifrice is selected for envelopably charging within a relatively-thin readily-masticatory and hydroscopic/salivascopic material, and such material being preferably of a conventional normally-solid thermoplastic gelatinous type, and being readily commercially available in: chargeably amenable capsule form, in thin-sheet form, and the like. A plurality of such dentifrice-enveloped articles might be assembled into a storage vessel and for periodic retrieval therefrom whereby, at subsequent and chronologically finite teethbrushing periods, the user can orally masticably release the envelopically charged dentifrice.

### BRIEF DESCRIPTION OF THE DRAWING

In the drawing, wherein like characters refer to like parts in the several views, and in which:

FIG. 1 is a top plan view of a representative embodiment 9A of the “Masticatory-Releasable Dentifrice Article” of the present invention;

FIG. 2 is a sectional elevational view of the representative embodiment 9A taken along lines 2-2 of FIG. 1;

FIG. 3 is a top plan view, reminescent to FIG. 1, of a representative embodiment 9B of the “Masticatory-Releasable Dentifrice Article” of the present invention;

FIG. 4 is a sectional elevational view of the representative embodiment 9B taken along lines 4-4 of FIG. 3;

FIG. 6 is a schematic flow-sheet diagram indicating processing steps for toothbrushing techniques utilizing the representative embodiments 9A and 9B mentioned hereabove ; and

FIG. 5 is a sectional elevational view showing the “Masticatory-Releasable Dentifrice Article” concept of the present invention embodied into an Articles manually-retrievable Articles-Container.

### DETAILED DESCRIPTION OF THE DRAWING

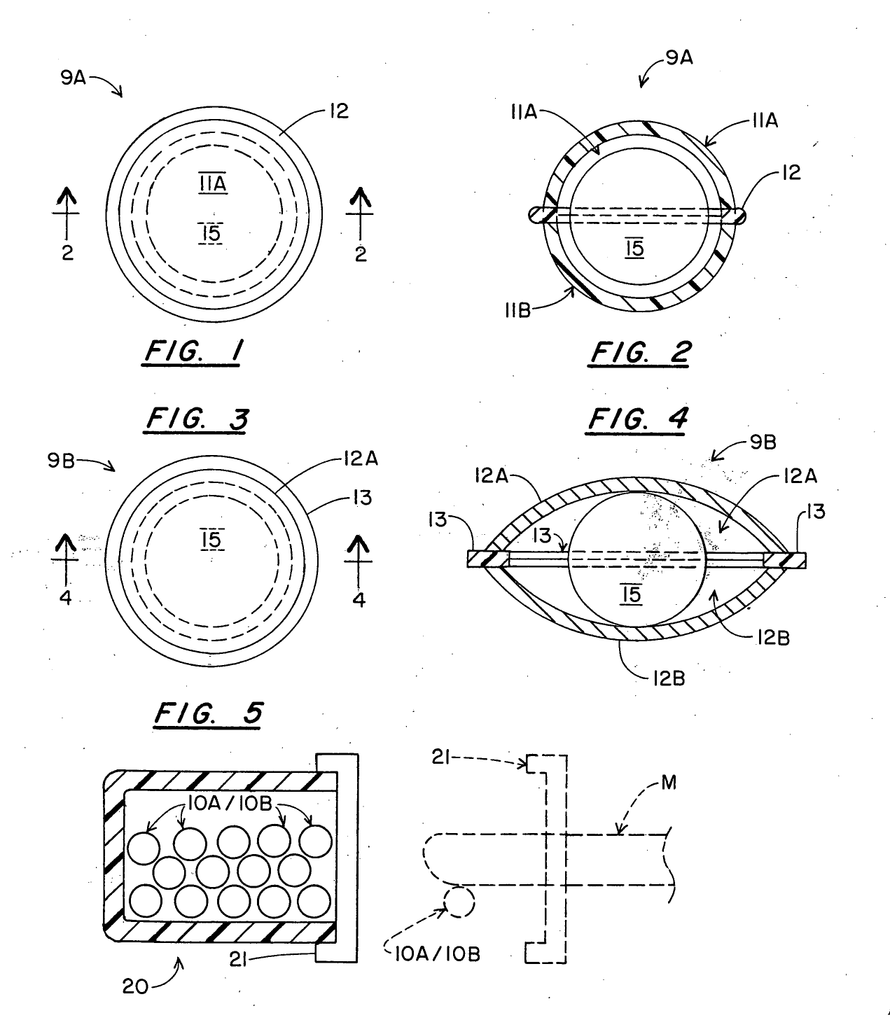

Drawing FIGS. 1 and 2 are directed toward a representative embodiment 10A for the “Masticatory-Releasable Dentifrice Article” concept of the present invention, and envelopably surrounding therewithin (11A, 11B, 12) a single teethbrushing dosage (15) of selectable readily commercialy available semi-solid-plastic and/or powdered-solid dentifrice (15). Such dentifrice encapsulated envelopment for embodiment 10A is furnished by globularly/spherically-shaped, hydrophilic/salivascopic material, such as from rudimentary gelatinous materials readily, inter alia, commercially available in thin-walled semi-hemispheric forms. In the latter regard, the reader's attention is respectfully brought to Volume 3 (pages 464-465) of the 1998 reference work entitled “How Products are Made” published by Gate Research, 835 Penobscot Building, Detroit, Mich. 48226-4094. Consistent with the technological teachings of said reference work, said embodiment 10A globular envelope (surrounding a dentifrice dosage 15) includes thin-walled semi-spheres 11A and 11B conventionally thermoplastically circularly peripherally joined at 12.

Drawing FIGS. 3 and 4 are directed toward an alternative representative embodiment 10B for the “Masticatory-Releasable Dentifrice Artice” concept of the present invention, and envelopably surrounding (12A, 12B, 13) a same type selectable dentifrice (15). This alternative envelpe encapsulation comprises two, normally laminar and flexible sheets (12A, 12B) of readily commercially available hydrophilic/salivaphilic thermoplastic gelatinous laminae (12A, 12B) which are thermoplastically joined (13) about dentifrice dosage (15).

As a further embellishment for the representative embodiments (e.g. 10A, 10B, etc.) of the “Masticatory-Releasable Dentifrice Articles” concept of the present invention alluded to hereabove, these and other representative embodiments might be plurally assembled together as two laminar gelatinous sheets (12A, 12B) which are (peripherally about a selectable dentifrice (15)) thermoplastically joined (13).

Consistent with the disclosures made in the three immediately preceeding paragraphs, the reader's attention is brought to the attached drawing FIG. 6 which method-wise schematically indicates utilization of the “Masticatory-Releasable Dentifrice Articles” e.g. 10A, 10B, etc., and until final teeth-brushing utilization.

As an intermediate method step, for the “Masticatory-Releasable Dentifrice Articles” of the present invention, representative “Articles” e.g. 10A, 10B, etc., might be plurally gathered into an interim storage vessel 20 (alluded to in FIG. 5 generally) and whereby single such “Articles” might be individually digitally manually (M) withdrawn therefrom via a removable cap (21) for the periodic teeth-brushing sessions alluded to in schematically indicated method steps (FIG. 6).

From the foregoing, the “Masticatory-Releasable Dentifrice Articles” concept of the present invention will be readily understood and further explanation is believed to be unnecessary. However, it is not desired to limit the invention to the-exact modes described hereabove, and accordingly, all suitable modifications and equivalents might be resorted to falling within the scope and penumbra of the appended claims.

1. Masticatory-Releasable Dentifrice Article comprising:
(A) a teethbrushing session dosage of selectable dentifrice; and
(B) said dosage being initially confined within a surrounding envelope of relatively-thin readily-masticatable and conventional hydrophilic/salivascopic material to provide a said Dentifrice Article and thereby enabling a teethbrushing person to serially: charge a said Dentifrice Article into one's oral cavity; then to masticate said Dentifrice Article to thereby release said dentifrice dosage; and finally enabling conventional teethbrushing by said released dentifrice dosage.

2. The Masticatory-Releasable Dentifrice Article of claim 1 wherein the said surrounding envelope is constructed throughout of a conventional hydrophilic/salivascopic and safely ingestible gelatinous material.

3. The Dentifrice Article of claim 2 wherein the surrounding envelope is of globular shape and along an axis thereof is of circularly annular shapes.

4. The Dentifrice Article of claim 3 wherein the temporarily confined dentifrice dosage is of the semi-solid plastic type.

5. The Dentifrice Article of claim 3 wherein the temporarily confined dentifrice-dosage is of the powdered-solid type.

6. The Dentifrice Article of claim 3 upgraded into an Articles Container wherein there is a plurality of said Dentifrice Articles storably charged within a removably-capped vessel, whereby a teethbrushing person might withdraw one or more of said plurality at a single teeth-brushing session.

7. The Masticatory-Releasable Dentifrice Article of claim 2 wherein the dentifrice dosage surrounding envelope in plan view is of polygonal shape and comprises two parallel and opposed layers of a said gelatinous material, and said two layers being fused at the said polygonal periphery in surrounding relationship to said dentifrice dosage.

8. The Masticary-Releasable Dentifrice Article of claim 7 wherein the temporarily confined dentifrice dosage is of the semi-solid plastic type.

9. The Masticatory-Releasable Dentifrice Article of claim 7 wherein upgraded into an Articles-Container wherein there is a plurality of said Dentifrice Articles storably charged within a removably-capped vessel, whereby a teethbrushing person might withdraw one or more of said plurality Articles at a single teethbrushing session.
